(19) United States
(12) Patent Application Publication
(10) Pub. No.: US 2026/0053440 A1
(43) Pub. Date: Feb. 26, 2026

(54) INTEGRATED SMART GARMENT

(71) Applicant: W. L. Gore & Associates, Inc., Newark, DE (US)

(72) Inventor: Hailey Felts, North East, MD (US)

(21) Appl. No.: 19/100,870

(22) PCT Filed: Aug. 2, 2023
(86) PCT No.: PCT/US2023/029302

§ 371 Date: Feb. 3, 2025

Related U.S. Application Data
(60) Provisional application No. 63/395,192, filed on Aug. 4, 2022.

(51) Int. Cl. A61B 5/00

(52) U.S. Cl. CPC A61B 5/6804; A61B 2562/06; A61B 2562/222

(57) ABSTRACT

A smart garment includes a garment and a sensor harness attached to the garment. The garment includes an electronic component attachment structure such as a pocket configured to receive an electronic component in a releasable manner. The sensor harness includes an electrical connector component configured to be mechanically and electrically coupled in a releasable manner to an electronic component attached to the garment by the electronic component attachment structure.

### CROSS-REFERENCE TO RELATED APPLICATION

This application is a national phase application of PCT Application No. PCT/US2023/029302, internationally filed on Aug. 2, 2023, which claims the benefit of Provisional Application No. 63/395,192, filed Aug. 4, 2022, which are incorporated herein by reference in their entireties for all purposes.

### FIELD

The present disclosure relates generally to apparel or garments that include electronic components such as sensors.

### BACKGROUND

Wearable electronics and smart garments or apparel are becoming increasingly popular. These smart garments, which include sensors and other electronic components, can be used to collect a wide range of information about the user wearing the garment. Examples of such information include physiologic information, such as the pulse rate and oxygen saturation of a wearer, and ergonomic or movement information.

There remains, however, a continuing need for improved smart garments and associated components. In particular, there is a continuing need for smart garments and components that are durable, convenient to use, and capable of accurately providing information about the user.

### SUMMARY

Smart garments in accordance with the disclosed examples include a sensor harness attached to a garment and may provide a number of advantages. For example, in addition to facilitating the accurate collection of physiologic, movement and other information from the user, the garment may be comfortable to wear, and not constrain movement of the user. The garment includes an attachment structure that facilitates the convenient and secure attachment of an electronic component that may be attached to the sensor harness. The garment is durable and suitable for repeated machine washing with the sensor harness attached thereto. The sensor harness can be conveniently removed from the garment at the end of life, for example recycling.

According to one example (“Example 1”), A smart garment, including a garment configured to be worn by a user, including a trunk portion and first and second limb portions extending from the trunk portion, wherein the first limb portion is configured to cover at least a portion of a first limb of the user, and the second limb portion is configured to cover at least a portion of a second limb of the user; and an electronic component attachment structure configured to receive an electronic component in a releasable manner to a location on the trunk portion; and a sensor harness attached to the garment, including: a plurality of wires; an electrical connector component coupled to the plurality of wires, wherein the electrical connector component is configured to be mechanically and electrically coupled in a releasable manner to an electronic component attached to the garment by the electronic component attachment structure; and a plurality of sensors, including at least a first sensor, a second sensor, and a third sensor coupled to the plurality or wires, wherein the first sensor is located on the first limb portion, the second sensor is located on the second limb portion, and the third sensor is located on the trunk portion.

According to another example (“Example 2”), further to Example 1, wherein the electronic component attachment structure includes one or more of a pocket, a tunnel, a snap, a hook and loop fastener, a mount, stitching, gluing, lamination, welding, or a combination thereof.

According to another example (“Example 3”), further to Example 1, wherein the electronic component attachment structure includes a pocket and the pocket opens to an interior or an exterior of the trunk portion.

According to another example (“Example 4”), further to Example 1, wherein the electronic component attachment structure includes a tunnel, the tunnel including two openings; the first opening to an exterior of the trunk portion; and a connector opening that opens to the interior of the trunk portion, wherein a portion of the plurality of wires and the electrical connector component extend through the connector opening into the tunnel.

According to another example (“Example 5”), further to Example 4, wherein the connector opening opens into the tunnel.

According to another example (“Example 6”), further to any of Examples 1-5, including a sensor cover that is configured to cover over one or more of the plurality of sensors on an interior of the garment.

According to another example (“Example 7”), further to Example 6, wherein the sensor cover is configured to attach the associated one or more of the plurality of sensors to the garment.

According to another example (“Example 8”), further to Example 6 or 7, wherein the sensor cover defines a tunnel in which the associated one or more of the plurality of sensors is located.

According to another example (“Example 9”), further to any of Examples 1-8, including a harness cover that is configured to cover over at least a portion of the plurality of wires.

According to another example (“Example 10”), further to Example 9, wherein the harness cover defines a tunnel in which the associated portions of the plurality of wires are located.

According to another example (“Example 11”), further to any of Examples 1-10, including releasable fasteners, optionally snap fasteners, attaching (1) one or more of the plurality of sensors, and/or (2) the plurality of wires, to the garment.

According to another example (“Example 12”), further to any of Examples 1-11, wherein the trunk portion includes a first portion, configured to extend around a trunk of the user when worn, and wherein the first portion has a first diameter when in an unstretched state; and a second portion attached to the first portion and configured to extend around the trunk of the user when worn, wherein the second portion has a second diameter that is less than the first diameter when in an unstretched state, and wherein the electronic component attachment structure is on the second portion of the trunk portion.

According to another example (“Example 13”), further to Example 12, wherein the first portion of the trunk portion includes a first elasticity in a circumferential direction; and the second portion of the trunk portion includes a second elasticity in a circumferential direction that is greater than the first elasticity.

According to another example (“Example 14”), further to any of Examples 1-11, wherein the trunk portion includes a first portion configured to extend around a trunk of the user when worn, and wherein the first portion has a first elasticity in a circumferential direction; and a second portion attached to the first portion and configured to extend around the trunk of the user when worn, wherein the second portion has a second elasticity in a circumferential direction that is greater than the first elasticity, and wherein the electronic component attachment structure is in or on the second portion of the trunk portion.

According to another example (“Example 15”), further to any of Examples 12-14, wherein the garment is an upper body garment; the trunk portion is configured to be worn on the user's torso; the first portion of the trunk portion is configured to cover an upper portion of the user's torso; the second portion of the trunk portion is configured to cover at least a portion of a lower portion of the user's torso, including the user's waist and/or hips; and the first and second limb portions comprise first and second sleeves.

According to another example (“Example 16”), further to any of Examples 1-11, wherein the garment is an upper body garment; the trunk portion is configured to be worn on the user's torso; and the first and second limb portions comprise first and second sleeves.

According to another example (“Example 17”), further to any of Examples 15-16, wherein the sensor harness includes a first torso portion extending transversely across the trunk portion, across and between shoulders of the user when the garment is worn, and wherein the third sensor is on the torso portion of the sensor harness; a second torso portion extending longitudinally down the trunk portion from the first torso portion, and wherein the electrical connector component is located on an end of the second torso portion opposite the first torso portion; a first sleeve portion extending from the first torso portion onto the first sleeve, and wherein the first sensor is on the first sleeve portion; and a second sleeve portion extending from the first torso portion onto the second sleeve, and wherein the second sensor is on the second sleeve portion.

According to another example (“Example 18”), further to any of Examples 1-11, wherein the sensor harness includes a torso portion extending transversely across the trunk portion of the garment, and wherein the third sensor is on the torso portion of the sensor harness; a first limb portion extending from the torso portion onto the first limb portion of the garment, and wherein the first sensor is on the first limb portion; and a second limb portion extending from the torso portion onto the second limb portion of the garment, and wherein the second sensor is on the second limb portion.

According to another example (“Example 19”), further to any of Examples 1-18, wherein the sensor harness further includes a flexible, and optionally stretchable, and optionally elastic, base, wherein the plurality of wires are fixed to the flexible base.

According to another example (“Example 20”), further to any of Examples 1-19, wherein the plurality of sensors comprise one or more of an accelerometer, a magnetometer, a gyroscope, a wireless communication device, a heart rate sensor, a sweat sensor, a glucose monitor, and electrocardiogram (ECG) sensor, a blood oxygen sensor, a temperature sensor, a pressure sensor, an atmospheric oxygen sensor, an atmospheric quality sensor, a humidity sensor, a noise level sensor, or a global positioning sensor (GPS).

According to another example (“Example 21”), further to any of Examples 1-20, further including an electronic component attached to the garment by the electronic component attachment structure and coupled to the sensor harness via the electrical connector component.

According to another example (“Example 22”), further to any of Examples 1-21, wherein the smart garment is water resistant or waterproof.

According to another example (“Example 23”), further to Example claim 22, wherein the garment is configured to be machine washable when the sensor harness is attached to the garment, and no electronic component is received by the connector plug.

According to another example (“Example 24”), further to any of Examples 1-21, wherein the garment is configured to facilitate removal of the sensor harness from the garment.

According to another example (“Example 25”), further to any of Examples 1-21, wherein at least portions of the sensor harness are not visible from an exterior of the garment.

According to another example (“Example 26”), a smart garment including a garment configured to be worn by a user, includes a trunk portion and first and second limb portions extending from the trunk portion, wherein the first limb portion is configured to cover at least a portion of a first limb of the user, and the second limb portion is configured to cover at least a portion of a second limb of the user; and an electronic component attachment structure configured to receive an electronic component in a releasable manner to a location on the trunk portion; and a sensor harness attached to the garment, including: a flexible base; a plurality of wires; an electrical connector component coupled to the plurality of wires, wherein the electrical connector component is configured to be mechanically and electrically coupled in a releasable manner to an electronic component attached to the garment by the electronic component attachment structure; and a plurality of sensors, including at least a first sensor, a second sensor, and a third sensor coupled to the plurality or wires, wherein the first sensor is located on the first limb portion, the second sensor is located on the second limb portion, and the third sensor is located on the trunk portion wherein the plurality of wires is attached to the flexible base, wherein the trunk portion includes a first portion configured to cover an upper portion of the torso, and a second portion configured to cover a lower portion of the torso; and wherein the electrical attachment structure is located on the second portion.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIGS. 1A and 1B are diagrammatic illustrations of the front and back sides, respectively, of a smart garment in accordance with embodiments.

FIGS. 2A and 2B are detailed diagrammatic illustrations of the inside and outside, respectively, of a portion of a smart garment including a pocket for receiving an electronic component, in accordance with embodiments.

FIG. 3 is a diagrammatic illustration or a sensor harness for a smart garment, in accordance with embodiments.

FIG. 4 is a diagrammatic illustration of a portion of a smart garment, including tunnel structures attaching a sensor harness to a garment, in accordance with embodiments.

FIG. 5 is diagrammatic illustration of a snap fastener structure and portions of a sensor harness, that can be used to attach the sensor harness to a garment, in accordance with embodiments.

FIG. 6 is an isometric illustration of an electronic component including a connector socket that can be used with a smart garment, in accordance with embodiments.

FIG. 7 is an isometric illustration of a portion of a sensor harness including a connector plug that can be used with a smart garment, in accordance with embodiments.

FIG. 8 is an isometric illustration of a connector plug mechanically and electrically coupled to a connector socket of an electronic component, in accordance with embodiments.

FIG. 9 is a diagrammatic illustration of electronics incorporated into an electronic component, in accordance with embodiments.

FIGS. 10A and 10B are illustrations of the front and back sides, respectively, of a smart garment in accordance with embodiments.

FIG. 11 is a detailed illustration of a portion of a smart garment including a tunnel for receiving an electronic component, in accordance with embodiments.

### DETAILED DESCRIPTION

The disclosures of all cited patent and non-patent literature are incorporated herein by reference in their entirety.

As used herein, the term “embodiment” or “disclosure” is not meant to be limiting, but applies generally to any of the embodiments defined in the claims or described herein. These terms are used interchangeably herein.

Unless otherwise disclosed, the terms “a” and “an” as used herein are intended to encompass one or more (i.e., at least one) of a referenced feature.

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. In addition, references to the singular may also include the plural (for example, “a” and “an”may refer to one or more) unless the context specifically states otherwise.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

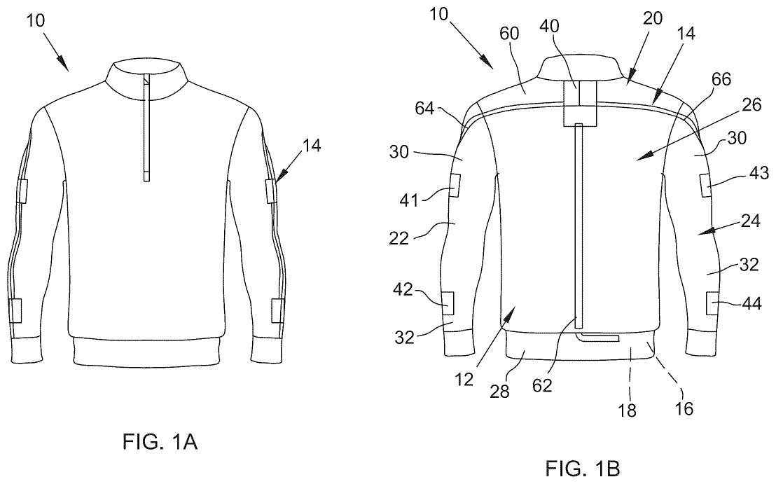

FIGS. 1A and 1B illustrate the front and back sides, respectively, an exemplary smart garment 10 in accordance with embodiments. The smart garment 10 includes a garment 12 and a sensor harness 14. As described in greater detail below, the sensor harness 14 includes an attachment structure 16 that is configured to receive, and to hold or attach to the garment, an electronic component 18 that can be coupled to the sensor harness 14. The attachment structure 16 is configured to receive the electronic component 18 in a releasable manner, so as to facilitate the attachment of the electronic component to the garment and the removal of the electronic component from the garment. The electronic component 18 is configured to be coupled to the sensor harness 14 in a releasable manner so as facilitate electrical and mechanical connection of the electronic component to the sensor harness and the disconnection of the electronic component from the sensor harness. When the electronic component 18 is attached to the garment 12 and coupled to the sensor harness 14, and the smart garment 10 is being worn by a user, the sensor harness captures data, such as motion data and/or physiologic data, of the user, and couples the collected data to the electronic component (e.g., for storage and/or transmission). The electronic component 18 can be conveniently disconnected from the sensor harness 14 and removed from the garment 12. In embodiments, the garment 12 is water resistant or waterproof. The smart garment 10, including the garment 12 and sensor harness 14 attached thereto, can therefore be washed or otherwise cleaned when the electronic component 18 is removed. The sensor harness 14 can be water resistant or waterproof, as defined by the capability of suitable performance following at least fifty 40° C. wash/warm tumble dry machine washes. The smart garment is configured to be washable, for example, when the sensor harness 14 is attached to the garment and no electric component is received by the connector plug 46. The sensor harness 14 and its location on the garment 12 facilitate the accurate capture of the user data. In further embodiments, at least portions of the sensor harness are not visible from an exterior of the garment.

Garment 12 is a shirt or similar apparel item configured to cover at least portions of the user's upper body or torso in the illustrated embodiments. However, other embodiments include other types of garments, including apparel such as pants, configured to cover at least portions of the user's lower body. The illustrated garment 12 includes a trunk portion 20 and first and second limb portions 22 and 24, respectively, extending from the trunk portion. Trunk portion 20 is configured to extend over or around all or portions of the user's toro when worn, and in the illustrated embodiments includes a first portion 26 configured to cover an upper portion of the torso, such as for example the shoulders and/or chest, and a second portion 28 configured to cover a lower portion of the torso, such as for example the waist and/or hips (e.g., an area around the user's pelvis). The first and second limb portions 22 and 24 are long sleeves in the illustrated embodiments, and include a first or upper arm portion 30 and a second or lower arm portion 32. The upper arm portions 30 of the first and second limb portions 22 and 24 are configured to cover at least portions of the user's upper arms, and the lower arm portions 32 are configured to cover at least portions of the user's lower arms. Although FIGS. 1A and 1B illustrate a long sleeve shirt, other embodiments of garment 12 include a short sleeve shirt (e.g., a shirt that does not include lower arm portions 32. In some embodiments, the smart garment 10 can be water resistant or waterproof.

In embodiments, the garment 12 may be formed from one or more textile panels or members, each including one or more layers of material, by conventional or otherwise known approaches. Stitching, sewing, lamination, heat bonding and adhesive bonding are examples of methods by which the panels or members of the garment 12 may be attached to one another. For example, each of the first and second limb portions 22 and 24 may be formed separately from one or more material members separate from the trunk portion 20 by approaches including stitching, sewing, gluing, laminating, or welding and attached to the trunk portion by stitching, sewing, gluing laminating, or welding. The material of the first and second limb portions 22 and 24 may be the same as or different than the material of the trunk portion 20. The trunk portion 20 may be formed by one or more material members having one or more layers. In embodiments, for example, the first portion 26 and the second portion 28 of the trunk portion 20 are formed separately from one or more material members by approaches such as stitching, sewing, gluing, laminating, or welding and are attached to one other by approaches such as stitching, sewing, gluing, laminating, or welding.

Non-limiting examples of suitable materials from which the garment 12 can be formed include woven, knitted or nonwoven fabric or textile substrates made from natural or synthetic fibers, filaments, yarns or any combination thereof. For example, the textile substrate may be produced from natural materials such as for example wool, cotton, silk, flax, hemp, jute, sisal, cellulose. Alternatively or additionally, the material may be produced from polymeric materials, optionally one or more of silicone; polyurethanes; polyesters, for example, polyethylene terephthalate, polytrimethylene terephthalate and/or polybutylene terephthalate; polyamide, for example, nylon 6, nylon 6,6; polyaramids, for example, NOMEX® aramid or KEVLAR® aramid, acrylic; fluoropolymers, for example, polytetrafluoroethylene (PTFE), expanded polytetrafluoroethylene (ePTFE); acrylates; methacrylates; polyethers; polyesteramides, polyetheramides; polyetheresters; polyetherurethanes; polyesterurethanes; or polyetheresterurethanes; or copolymers, blends or multilayer laminates thereof. In some embodiments, the textile substrates can be flexible, stretchable and/or elastic and/or they may be breathable, having a moisture vapor transmission rate of >1000 g/m2/day and <50,000 g/m2/day (MVTR test disclosed in DIN EN ISO 15496 (2004)). As used herein, the term elastic means a substrate that can be stretched to a length of greater than or equal to 5% of its original non-tensioned length and has greater than or equal to 90% recovery of its original length when the tension is released. In other embodiments, the substrate can be stretched to about 100% of its original non-tensioned length and has at greater than or equal to 80% recovery of its original length when the tension is released. Conventional or otherwise known weaving, knitting, crocheting, knotting, tatting, felting, banding or braiding processes are nonlimiting examples of methods that may be used to produce the material of the textile embodiments of substrate 118.

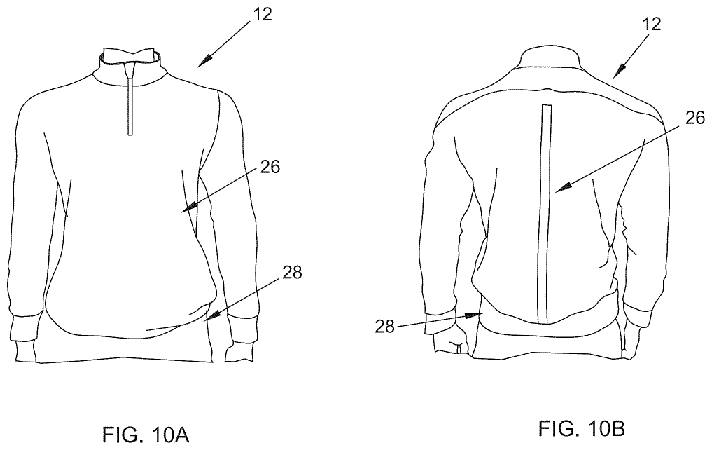

In embodiments, the garment is an upper body garment (e.g., a shirt) comprising a trunk portion, wherein the first portion 26 and the second portion 28 of the trunk portion 20 have different characteristics, such as for example to provide different fit characteristics when worn by the user. For example, the sizing and/or elastic properties of the second portion 28, or at least portions of the second portion 28, may be different than the sizing and/or elastic properties of the first portion 26 so that the second portion 28, including or at least the pocket 16, fits more closely and snugly to the waist and/or hips of the user than the fit of the first portion 26 to the chest of the user. In some embodiments, the first portion 26 is configured to extend around a trunk of the user when worn, and the first portion 26 has a first diameter when in an unstretched state; and the second portion 28 attached to the first portion is also configured to extend around the trunk of the user, and the second portion 26 has a second diameter that is less than the first diameter when in an unstretched state. FIGS. 10A and 10B, for example, illustrate embodiments of the garment 12 having a first portion 26 sized and shaped to fit relatively loosely on portions of the user's body, including slack portions that are not under tension, for example when the user's arms are not extended. The second portion 28, may be sized and shaped to fit relatively tighter than the first portion 26, and may be under tension around the hips of the user. Potentially distracting or otherwise unwanted movement of the electronic component 18 may thereby be prevented or minimized. Upper portion 26 and/or the first or second limb portions 22 and 28 may be sized to provide sufficient comfort when worn by the user as to not substantially constrain or interfere with their motion, yet sufficiently snug that the sensor harness 14 is sufficiently accurately positioned with respect to the user's body to facilitate the accurate collection of the motion data. In embodiments of these types the second portion 28 may have a diameter in an unstretched state that is smaller than a diameter of the first portion 26 when in an unstretched shape. Alternatively or additionally, the elasticity of the second portion 28 (e.g., in a circumferential or diametric direction that causes the second portion 28 to compress onto the torso of the user) may be greater than an elasticity of the first portion 26.

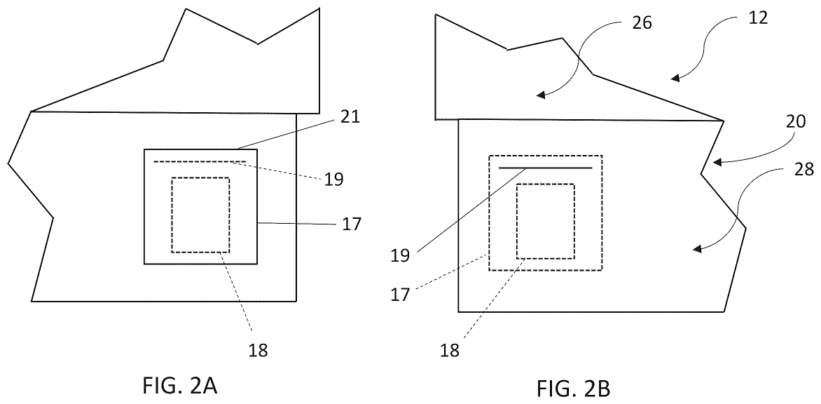

FIGS. 2A and 2B are detailed illustrations of outside and inside portions, respectively of the trunk portion 20 of the garment 12, including an attachment structure 16 in the form of a tunnel 17. In some embodiments, the electronic attachment structure comprises a pocket having one opening to an interior or an exterior of the trunk portion. In other embodiments, the electronic attachment structure comprises a tunnel having at least two openings, to the interior of the garment, to the exterior of the garment or to both the interior and the exterior of the garment. In the illustrated embodiments the tunnel 17 is formed by a piece of material attached to the second portion 28 of the trunk portion(e.g., by stitching, lamination, gluing or welding). The tunnel 17 includes an opening, such as for example at its upper end 21, that is sized to facilitate the insertion and removal of the electronic component 18. A slot 19 through the material of the garment 12 may be used to provide access to the tunnel 17 for components of the sensor harness 14, and in the illustrated embodiments opens into the interior of the tunnel 17. The slot 19 may be oriented parallel to the opening, or it may be oriented in any direction relative to the opening, including oriented about 180° to the opening, which may facilitate insertion of components of the sensor harness into the tunnel 17. As described in greater detail below, a portion of the sensor harness 14 can extend from the inside of the garment 12 through material of the garment and into the tunnel 17, facilitating the connection of the sensor harness to the electronic component 18. The configuration of the garment 12 shown in FIGS. 2A and 2B allows both the sensor harness 14 and the electronic component 18 to be covered and shielded from view from the outside of the garment when the garment is worn by a user. In other embodiments, the garment comprises a pocket, that is attached to the trunk portion on at least one side, with at least a portion of the pocket open to receive the sensor harness, the electronic component or both. The sensor harness is coupled to the electronic component and the coupled sensor harness/electronic component are placed into the pocket where they are securely held during use.

Although shown at a particular (e.g., back and side) location on the garment 12 in FIGS. 2A and 2B, in other embodiments the attachment structure 16 is positioned at other locations on the garment 12 (e.g., at other locations on the back side, or other locations such as on the front side of the garment). An advantage of positioning the attachment structure 16 on the outside of the garment 12 is that it facilitates convenience of access to, and the attachment and removal of, the electronic component 18 by the user. In other embodiments, the attachment structure 16 is positioned at other locations, such as on the inside of the garment 12, or between multiple layers of material forming the garment.

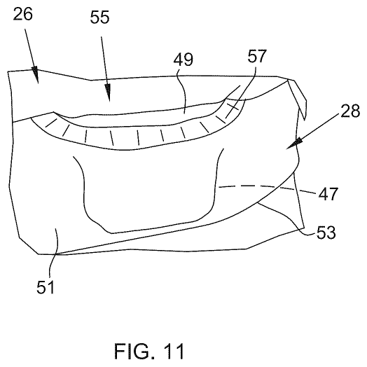

FIG. 11 illustrates an attachment structure 16 comprising a tunnel 47 in the second portion 28 of the garment 12. In the illustrated embodiments, the tunnel 47 is defined by two sections of material 49 and 51 that overlay one another to form the tunnel between the sections of material. For example, one or more pieces of material may be folded at an edge 53 (e.g., that defines the bottom of the garment 12), and sides of the pieces of material opposite the edge 53 may be attached to the first portion 26 of the garment. In embodiments, the tunnel 47 may extend circumferentially around all or portions of the second section 28 of the garment 12. The tunnel comprises an opening 55 to the exterior of the trunk portion and may be used to provide access to the tunnel 47. The trunk portion may further include a connector opening wherein a portion of the plurality of wires and the electrical connector component extend through the connector opening into the tunnel. In the embodiment shown in FIG. 11, for example, the opening 55 is defined by an edge portion 57 of the section of material 51 that is not attached to the first portion 26 of the garment 12. In some embodiments, the openings or slots of the pockets and/or tunnels may be elasticized to help retain the sensor harness and/or the electronic component.

Although shown for purposes of example as a tunnel 17 in FIGS. 2A and 2B and a tunnel 47 in FIG. 11, attachment structure 16 can take other forms in other embodiments. For example, alternatively or additionally, the attachment structure 16 may include one or more of a snap fastener component, a component of a hook and look fastener, adhesive, clip, belt, stitching, a pillowcase flap, an inner surface of a non-slip material, a zipper, or other mount or fastener, or a combination thereof. In other embodiments, the tunnel, the pocket, or other parts of the can comprise an anti-static and/or electronic shielding material.

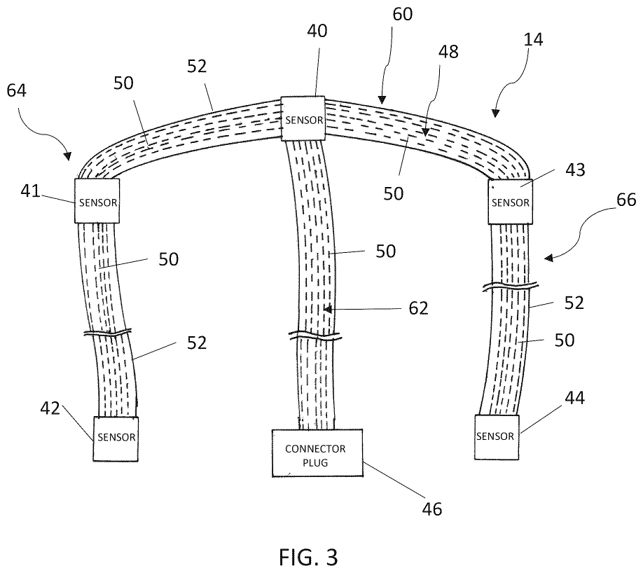

FIG. 3 and FIGS. 1A and 1B illustrate the sensor harness 14. As shown, sensor harness 14 includes a plurality of sensors 40, 41, 42, 43 and 44 arranged in a satellite configuration with respect to a connector plug 46. The sensors 40-44 are physically and electrically coupled to the connector plug 46 by a conductive webbing or electrical cable 48. Cable 48 includes a plurality of flexible electrically conductive wires 50 supported by a flexible substrate or flexible base 52.

The illustrated embodiment of the sensor harness 14 is generally T-shaped when outstretched, and includes a first torso portion 60, second torso portion 62, first sleeve portion 64 and second sleeve portion 66. The first torso portion 60 is configured to extend generally transversely across the trunk portion 20 of the garment 12, and in the illustrated embodiments extends across the back side of the garment between the portions of the garment that cover the user's shoulders when the garment is worn. The first sensor 40 is shown positioned on the first torso portion 60 at a location that will be positioned adjacent to an upper and central location of the user's back, for example below the base of the user's neck, when the garment is worn. The second torso portion 62 is configured to extend generally longitudinally down the trunk portion 20 of the garment 12, and in the illustrated embodiments extends along the back side of the garment from the first torso portion 60 and the first sensor 40. The connector plug 46 is located on an end of the second torso portion 62 opposite the first torso portion 60. A length of the portion of the cable 48 of the second torso portion 62 of the sensor harness 14 is sufficiently long that the connector plug 46 can be located at the attachment structure 16 of the garment 12 when the garment is worn, with the connector plug 46 being retained at the attachment structure 16 without substantially constraining or interfering with motion of the user. First sleeve portion 64 and second sleeve portion 66 extend from the opposite sides or ends of the first torso portion 60, and are configured to extend along the limb portions 22 and 24, respectively, of the garment 12. In the illustrated embodiments the first and second sleeve portions 64 and 66 are configured to extend across the outer lateral sides of the limb portions 22 and 24, respectively, of the garment 12. The sensors 41 and 43 are positioned on the sleeve portions 64 and 66 of the sensor harness 14 at locations that will be positioned adjacent to the upper arms of the user, for example near the biceps, when the garment 12 is worn. The sensors 42 and 44 are positioned on the sleeve portions 64 and 66 of the sensor harness 14 at locations that will be positioned adjacent to the lower arms of the users, for example near the wrists, when the garment 12 is worn.

The sensor harness 14 can include multiple electrically conductive wires 50, such as, for example, individual wires, and/or a cable 48, for example, ribbon-type or bundled or twisted multi-conductor wires. In certain embodiments, the cable 48 and/or wires 50 may be extensible (e.g., define a serpentine path in two or more of x, y and z directions with respect to a plane of the flexible base 52) to accommodate flexibility and/or stretching of the flexible base 52. According to certain embodiments, the wires 50 may be positioned and/or attached on or adjacent the surface of the flexible base 52, within the thickness of the base, or within a tunnel or channel of the base. Additionally, the flexible base 52 and the harness 14 may be water resistant or waterproof, or otherwise configured to withstand machine washing or other cleaning.

Advantageously, the sensor harness 14 can retain conductive performance over a range of stretch and/or flex, thereby reducing the likelihood the cable becomes inoperable due to the stretching and/or flexing of the garment 12. For example, the sensor harness 14 can have negligible resistance change when stretched up to 50% strain of the original, relaxed configuration of the sensor harness 14. “Strain”, as defined herein, is meant to denote the extension of the flexible base 52 relative to its original, relaxed configuration. In some embodiments, the sensor harness 14 has negligible resistance change when stretched up to 100% strain or even over 100% strain. In some embodiments, the sensor harness 14 is more stretchable than the garment 12.

According to certain embodiments, the term “electrically conductive” as used herein with respect to the wires 50 is meant to describe a structure that provides a continuous line or continuous pathway that is able to conduct electrons therethrough. The wires 50 may be formed independently and separately from the base 50 and attached to the base. In exemplary embodiments, the wires 50 include insulating or non-conducting regions, such as for example dielectric coatings. Conventional or otherwise known cables 48 suitable for applications of the sensor harness 14 and/or garment 12 can be used as the electric conductor.

In certain examples, the plurality of wires 50 may be fixed or attached onto an outer surface of the flexible base 52 and/or knitted, woven, or otherwise incorporated into the thickness, or on or adjacent a surface of the flexible base 52. Non-limiting examples of suitable flexible bases 52 include woven, knitted or nonwoven textile substrates made from natural or synthetic fibers, filaments, yarns or any combination thereof. For example, the textile substrate may be produced from natural materials such as for example wool, cotton, silk, flax, hemp, jute, sisal, cellulose. Alternatively or additionally, the textile substrate may be produced from polymeric materials, optionally one or more of silicone; polyurethanes; polyesters, for example, polyethylene terephthalate, polytrimethylene terephthalate and/or polybutylene terephthalate; polyamide, for example, nylon 6, nylon 6,6; polyaramids, for example, NOMEX® aramid or KEVLAR® aramid, acrylic; fluoropolymers, for example, polytetrafluoroethylene (PTFE), expanded polytetrafluoroethylene (ePTFE); acrylates; methacrylates; polyethers; polyesteramides, polyetheramides; polyetheresters; polyetherurethanes; polyesterurethanes; or polyetheresterurethanes; or copolymers, blends or multilayer laminates thereof. In some embodiments, the textile substrates can be flexible, elastic and/or they may be breathable, having a moisture vapor transmission rate of >1000 g/m2/day and <50,000 g/m2/day (MVTR test disclosed in DIN EN ISO 15496 (2004)).

In certain examples, the wires 50 may be fixed or attached onto an outer surface of a flexible base 52 comprising a film or membrane. Non-limiting examples of suitable film or membrane flexible bases 52 include natural materials such as leather or fur, or polymeric materials, optionally one or more of silicone; polyurethanes; polyesters, for example, polyethylene terephthalate, polytrimethylene terephthalate and/or polybutylene terephthalate; polyamide, for example, nylon 6, nylon 6,6; polyaramids, for example, NOMEX® aramid or KEVLAR® aramid, acrylic; fluoropolymers, for example, polytetrafluoroethylene (PTFE), expanded polytetrafluoroethylene (ePTFE); acrylates; methacrylates; polyethers; polyesteramides, polyetheramides; polyetheresters; polyetherurethanes; polyesterurethanes; or polyetheresterurethanes; or copolymers, blends or multilayer laminates thereof. In some embodiments, the film or membrane substrates can be flexible, elastic and/or they may be breathable, having a moisture vapor transmission rate of >1000 g/m2/day and <50,000 g/m2/day (MVTR test disclosed in DIN EN ISO 15496 (2004)).

Elastic embodiments of the flexible base 52 can have greater than or equal to 5% stretch with at least 90% recovery. In other embodiments, the elastic substrate may have less than or equal to 100% stretch with at least 80% recovery. Embodiments of the flexible base 52 are woven, knitted or nonwoven textiles comprised of interlaced filamentary structures such yarns, threads or fibers. Conventional or otherwise known weaving, knitting, crocheting, knotting, tatting, felting, banding or braiding processes are nonlimiting examples of methods that may be used to produce the material of the textile embodiments of flexible base 52.

In certain embodiments, the wire 50 may be attached to the outer surface of the flexible base 52 to form the sensor harness 14. In exemplary embodiments, the flexible base 52 is flat (i.e., planar) and contains no wrinkles when the wires 50 are applied. The wires 50 may be attached such that the wire 50 is positioned on at least a portion of the outer surface of the flexible base 52 to form the sensor harness 14.

Embodiments include adhesive to attach all or portions of the wires 50 to the flexible base 52 (e.g., the adhesive is located between the electrically conductive wire and the base). Conventional or otherwise known adhesives suitable for the application of the wire 50 and flexible base 52 can be used to attach the electrically conductive wire to the surface of the base. Nonlimiting examples of such adhesives include polyurethane, polyester, polyolefin, epoxy, acrylate, methacrylates. In embodiments, the adhesive attaching the wires 50 to the flexible base 52 is located at discrete locations spaced apart from one another along a length of the electrically conductive wire. In other embodiments the adhesive extends continuously along the length of the wires 50 attached to the flexible base 52.

Alternatively or additionally, embodiments include potting to attach all or portions of the wires 50 to the flexible base 52 (e.g., the potting material extends from on one or more of the sides or top of the electrically conductive wire onto the base to at least partially overcoat or encapsulate portions of the wire and adjacent portions of the base and thereby secure the wire to the surface of the base). Conventional or otherwise known potting materials suitable for the application of the sensor harness 14 can be used to attach the electrically conductive wires 50 to the surface of the flexible base 52. Nonlimiting examples of such potting materials include the adhesives described above. In embodiments, the potting material attaching the electrically conductive wires 50 to flexible base 52 is located at discrete locations spaced apart from one another along a length of the electrically conductive wire. In other embodiments the potting material extends continuously along the length of the electrically conductive wire 50 attached to the flexible base 52.

Alternatively or additionally, embodiments include stitching to attach all or portions of the electrically conductive wire 50 to the flexible base 52. For example, filaments such as threads or wires can be wrapped over and around the electrically conductive wire and into or through the flexible base 52 by conventional or otherwise known stitching approaches. Yet other embodiments include other mechanical structures such as staples to fasten the wires 50 to the flexible base 52.

Alternatively or additionally, in embodiments all or portions of the electrically conductive wires 50 may be woven, knitted or otherwise inserted into the material of the flexible base 52 (e.g., located within the thickness of the substrate or on or adjacent a surface of the substrate) to attach the electrically conductive wire to the base. For example, in some instances, the wires 50 may be woven, knitted or otherwise inserted into a flexible base 52 during the fabrication of the textile substrate. In other instances, the electrically conductive wires 50 may be woven, knitted or otherwise inserted into the flexible base 52 after the fabrication of the base.

As used herein, “fixed” or “attached to” means that a wire 50 that is manufactured separately from the base 50 is joined to (e.g., on or adjacent a surface of and/or within the thickness of) or otherwise held or retained on the base. In at least some of the embodiments described above, for example, the electrically conductive wires 50 are “fixed” or “attached to” the flexible base 52 by processes that occur after and/or during the fabrication of the base.

In order to accommodate stretch and/or flexibility of the cable 48 and flexible base 52, the electrically conductive wires 50 may have characteristics that provide them with extensibility (e.g., to be effectively elongated) when portions of the base to which they are attached stretch and/or flex. In certain embodiments, for example, a wire 50 may define a zig-zag, sinusoidal or other serpentine path to effectively elongate and accommodate stretch and/or flex of the flexible base 52 to which it is attached. The zig-zag, sinusoidal, or other serpentine path may comprise the conductive wires with predictable and/or repeatable spacing and/or curvature. The curvature may be larger than the minimum bend radius of the conductive wire. The spacing may be sufficient to achieve desired electrical properties (such as for example impedance, capacitance, attenuation, cross talk, etc.). The sinusoidal paths of the wires 50 may be oriented about the x-y plane of the flexible base 52, and may be generally located on the surface of the base. In embodiments the wires 50 may be attached to the flexible base 52 by certain fibers of the base that overlay portions of the wires. Wires 50 may, for example, be attached to the flexible base 52 during the process by which the base is knitted or woven. Because of their serpentine nature, conductive wires 50 are effectively extensible and bendable, and may elongate and bend to accommodate stretching and/or flexing of the flexible base 52 to which they are attached.

Each of the sensors 40-44 is an electronic device configured to collect data from the user wearing the smart garment 10, and/or to provide electrical information representative of the data to the electronic component 18 via the wires 50 and connector plug 46. Sensors 40-44 may, for example, be configured to sense and collect user movement and/or position data, and/or user physiological data (e.g., pulse rate data, blood pressure data, oxygen saturation data, cardiac data and/or respiratory data), and/or environmental data (e.g., air temperature, humidity, air pressure). Nonlimiting examples of such sensors 40-44 include an accelerometer, a strain gauge, a biometric sensor, a temperature sensor, an ECG sensor, an EMG sensor, a blood oxygen sensor, and a blood glucose level sensor, a magnetometer, a gyroscope, a wireless communication device, a heart rate sensor, a sweat sensor, a pressure sensor, and atmospheric oxygen sensor, an atmospheric quality sensor, a humidity sensor, a noise level sensor, a global positioning sensor (GPS) or combinations thereof. Each sensor 40-44 may include a plurality of sensors configured to collect redundant and/or different types of data.

The sensors 40-44 are coupled to the connector plug 46 via one or more of the wires 50. Wires 50 may comprise any known or otherwise conventional structure for coupling electrical signals, including for example printed ink conductors and metal wires. Although five wires 50 are shown in FIG. 3 for purposes of example, other embodiments of sensor harness 14 may include more or fewer wires. For example, the sensor harness 14 may be configured with sufficient numbers of wires 50 to enable the operation of each sensor (e.g., to be powered as needed) and to couple the collected data to the connector plug 46. Although illustrated as including a multi-conductor cable 48, other embodiments of sensor harness 14 include one or more single wires such as 50.

Although the exemplary smart garment 10 and sensor harness 14 shown in FIGS. 1A, 1B and 3 have five sensors 40-44, other embodiments have more or fewer sensors. For example, a short sleeve embodiment of a smart garment 12 in the form of a shirt may not have sensors such as 42 and 44 configured to be positioned adjacent to the lower arms of the user. Embodiments may alternatively or additionally have one or more sensors positioned on the sensor harness 14 at locations such that when the sensor harness is mounted to the garment 12, a sensor is located adjacent to the lower back or wrists of the user wearing the garment.

Sensor harness 14 is preferably mounted to the garment 12 in manner that (1) positions the sensors 40-44 at desired predetermined locations on the garment so as to facilitate the accurate collection of data when the garment is worn, (2) is sufficiently secure to facilitate the washing or other cleaning of the smart garment without undue movement or detachment of portions of the sensor harness from the garment, and/or (3) enables the relatively easy or convenient removal of the sensor harness from the garment when such removal is desired (e.g., upon the disposal or recycling of the garment). Portions or all of the sensor harness 14 may be located on either or both of the inside or outside of the garment 12. In embodiments, portions or all of the sensor harness 14 may be located within a thickness of the material of the garment 12 (e.g., between two layers of material, or within a single layer of material). In embodiments, all or portions of the sensor harness 14 are covered by material, for example in a manner that minimizes or reduces visibility of the sensor harness 14 when the smart garment 10 is worn, and/or to enhance the visual appearance of the smart garment.

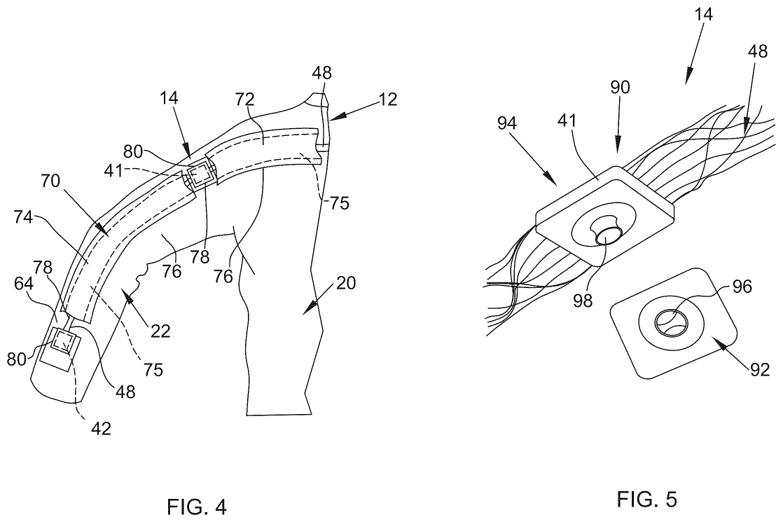

FIG. 4 is an illustration of a portion of a trunk portion 20 and first limb portion 22 of a garment 12 in the form of a long sleeve shirt, that includes a harness cover 70 for securing the first sleeve portion 64 of the sensor harness 14 to the garment. In the illustrated embodiments the harness cover 70 is formed from a plurality of cover tunnel sections 72 and 74 formed by material members that are attached to the garment 12 (e.g., by stitching 76 on the side edges of the material members). The cover tunnel sections 72 and 74 define a passageway, channel or tunnel 75 with the surface of the material of the torso portion 20. The harness cover 70 may include openings 78 at locations on the garment 12 (e.g., on the first limb portion 22) where the sensors such as 41 and 42 are located. For example, the opening 78 between the cover tunnel sections 72 and 74 is located at the desired position of the sensor 41, and the opening 78 at the end of the cover tunnel section 74 opposite the cover tunnel section 72 is located at the desired position of the sensor 42 in the illustrated embodiments. The embodiments illustrated in FIG. 4 include a sensor cover 80 covering over one or more of the sensors 41 and 42 to form a barrier between the sensors and the skin of the user wearing the garment. The sensor cover 80 may reduce any irritation or discomfort that might otherwise be caused by the sensors such as 41 and 42 being directly physically in contact with the skin of the user, and may also act as a barrier to minimize or otherwise reduce skin and other body tissue such as perspiration from contacting and potentially detrimentally impacting the operation of the sensors. In some embodiments, the sensor cover 80 is configured to attach the associated one or more of the plurality of sensors to the garment. This sensor cover 80 can help to reduce movement of the sensors, potentially providing more accurate motion data.

The tunnel 75 is sized to securely hold portions of the sensor harness 14 such as the cable 48 to the garment 12. The harness cover 70 may be formed before the sensor harness 14 is attached to the garment 12, and the tunnel 75 made sufficiently large to facilitate the insertion of the first sleeve portion 64 of the sensor harness 14 through the tunnel. In embodiments of these types, the sensor harness 14 may be detached or removed from the garment 12 by pulling the sensor harness through the harness cover 70. In other embodiments the harness cover 70 is attached to the garment 12 over the cable 48 and/or other portions of the sensor harness 14. The tunnel 75 may not be sufficiently large to receive the sensors such as 41 and 42 in these embodiments. In embodiments of these types the cover tunnel sections 72 and 74 may be removed from the garment 12 to provide openings for the sensor harness 14 to be removed from the garment. For example, stitching, or other structure such as adhesive or other bonds may be removed or broken to provide the opening for removal of the sensor harness 14. Alternatively or additionally, the cover tunnel sections 72 and 74 may be cut along their lengths to provide openings for removal of the sensor harness 14.

Although only harness cover 70 is shown in FIG. 4 for purposes of example, cover tunnel structures substantially the same as or similar to harness cover 70 may be formed on the garment 12 at appropriate locations to secure the first torso portion 60, second torso portion 62 and second sleeve portion 64 of the sensor harness 14 to the garment 12. For example, a harness cover such as 70 can be located on and extend longitudinally down the middle of the back of the garment 12 from near a collar to the bottom of the garment to secure the second torso portion 62 (e.g., as shown in FIG. 1B).

Tunnels such as 75 can also be formed by other structural arrangements. For example, one or more pin-tuck tunnels (not shown) may be formed from the material of the garment 12 by folding and doubling-back portions of the garment to form a two-layer flap extending in directions and at locations of the desired tunnels, and attaching the free-edge side of the flap and the edge of the flap on the side that meets the other portions of the material of the garment to the material of the garment (e.g., by stitching). The tunnel is thereby defined between the two layer flap and the adjacent portion of the garment 12. Openings may be formed in the material of the flap at the desired positions of the sensors. As yet another example, in garments 12 formed from multiple layers of material, the tunnel may be defined between the multiple layers of material (e.g., by stitching), and openings may be formed in the inner layer of material at the desired positions of the sensors. In some embodiments, each of the harness cover 70 and the sensor covers 80 can independently formed from a textile or from a polymeric encapsulant. In some embodiments, the harness cover 70 and the sensor cover 80 are made from the same textiles as the garment 10. In some embodiments, the harness cover 70 and the sensor cover 80 are made from textiles that are different from the garment 10.

FIG. 5 illustrates an exemplary snap fastener structure 90 that may be used to secure the sensor harness 14 to the garment 12. The snap fastener structure 90 includes a first fastener component 92 and a second, complimentary fastener component 94 that may be releasably mechanically coupled to one another. The first fastener component 92, which is shown as having a receiving member 96 in this example, is mounted to the garment 12. The second fastener component 94, which is shown as having a post 98 that is received by the receiving member 96 of the first fastener component 92 in this example, is mounted to the sensor harness 14. In the exemplary embodiment shown in FIG. 5, the second fastener component 94 is mounted to a sensor 41. The first fastener component 92 can be located on the surface of the garment 12 (not shown in FIG. 5) at a location, such as for example the upper arm portion 30 that it is desired to secure the sensor 41 to the garment. In other embodiments (not shown), one or more snap fastener structures such as 90 may be located at other corresponding positions on the garment 12 and sensor harness 14 (e.g., on the cable 48) to releasably secure those portions of the sensor harness to the garment at the desired locations.

Other embodiments of smart garment 12 include other types of releasable fastener structures having complimentary components (e.g., hook and loop fasteners) to mount the sensor harness 14 to the garment 12. One or more releasable fastener structures such as 90 may also be used in combination with one or more harness cover structures such as 70 described above. Alternatively or additionally, the sensor harness 14 can be attached to the garment 12 by other structures or approaches such as stitching, adhesive and/or staples (e.g., the cable 48 can be stitched to the material of the garment 12).

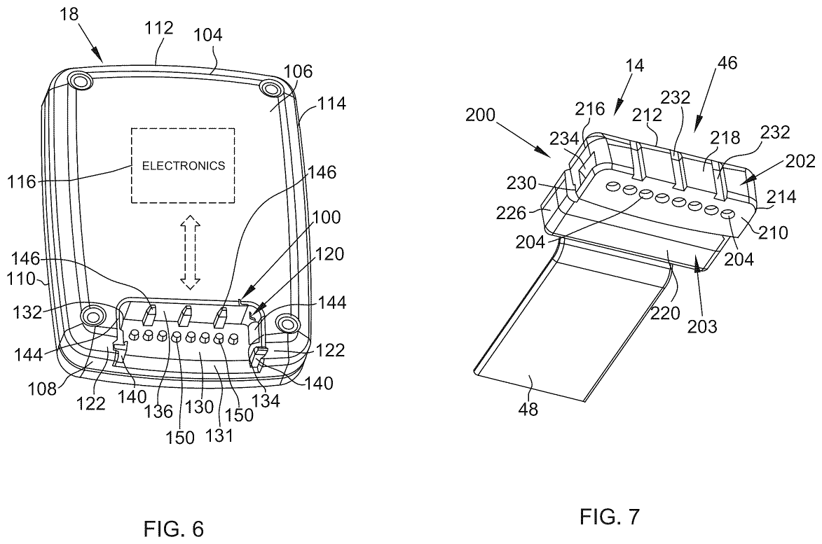

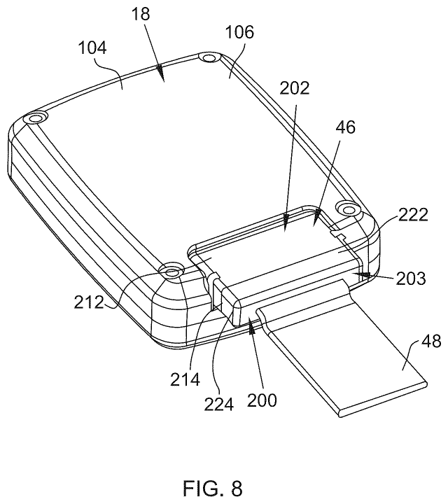

FIG. 6 is an isometric illustration of an electronic component 18 including a connector socket 100 in accordance with embodiments. FIG. 7 is an isometric illustration of a portion of the sensor harness 14 including the cable 48 and an embodiment of the connector plug 46 that can be mechanically and electrically coupled to the electronic component 18 in accordance with embodiments. FIG. 8 is an isometric illustration of the connector plug 46 mechanically and electrically coupled to the connector socket 100 of the electronic component 18. Electronic component 18, including the connector socket 100, and the connector plug 46 cooperate with one another to form an electrical connector system that can be incorporated into the smart garment 10.

Electronic component 18 includes a housing 104 having a first major wall 106, first, second, third and fourth side walls 108, 110, 112 and 114, respectively, and a second major wall (not visible in FIG. 6). The first, second, third and fourth side walls 108, 110, 112 and 114 have edge portions that are joined to and extend between edge portions of the first major wall 106 and second major wall to define a space within the housing 104. Electronics 116 within the housing 104 are electrically coupled to the connector socket 100.

The connector socket 100 includes a recess or receptacle 120 in the first major wall 106 of the housing 104. In the illustrated embodiments, the receptacle 120 is located adjacent to the side wall 108 of the housing 104, and also opens or extends into the housing from the side wall 108. The illustrated embodiments of the receptacle 120 thereby define an opening that extends into the housing 104 though the intersecting edge portions 122 of the first major wall 106 and the side wall 108. The receptacle 120 is generally rectangular in shape in the illustrated embodiments, and is defined by a bottom or receptacle contact wall 130, side walls 132 and 134, and an interior wall 136. The contact wall 130 extends from the side wall 108. Interior wall 136 extends between the first major wall 106 and an edge portion of the contact wall opposite the side wall 108. Side wall 132 extends between the side wall 108 and a side edge portion of the interior wall 136, and between the first major wall 106 and a side edge portion of the contact wall 130. Similar to the side wall 132, but on a side of the receptacle 120 opposite the contact wall 130 and interior wall 136, the side wall 134 extends between the side wall 108 and a side edge portion of the interior wall 136, and between the first major wall 106 and a side edge portion of the contact wall 130. In some embodiments, contact wall 130 may be essentially coplanar across the entirety of the surface of the contact wall 130. In other embodiments, contact wall 130 may have a lip or raised portion 131.

The illustrated embodiments of receptacle 120 include a lip or raised portion 131 at the edge of the receptacle adjacent side wall 108. The surface of the contact wall 130 may be recessed with respect to the raised portion 131. Raised portion 131 can mate with a complementary feature on connector plug 14 to help retain the electronic component 18 and the connector plug 46 in the coupled configuration. Other embodiments may not include structures such as the raised portion 131, and the surface of the contact wall 130 may be coplanar to the exterior of the housing 104.

One or more tabs 140 (two are shown for purposes of example in FIG. 6) extend into the receptacle from one or both of the receptacle side walls 132 or 134. In the illustrated embodiments, a tab 140 extends into the receptacle 120 from each of the receptacle side walls 132 and 134, at locations adjacent the housing side wall 108. Tabs 140 are generally rectangular in cross section, and extend substantially the entire height of the receptacle side walls or the entire depth of the receptacle 120 (e.g., in a direction between the first major wall 106 and the contact wall 130). As shown in FIG. 6, the tabs 140 are configured to define the receptacle 120 as including a space between the tabs in both the first major wall 106 and the housing side wall 108 in the illustrated embodiments. In other embodiments (not shown) the one or more tabs 140 can have other configurations such as shapes and sizes.

Embodiments of the connector socket 100 include springs 144 extending into the receptacle 120 from one or more of the receptacle walls 132, 134 and 136. The illustrated embodiments include two compression springs 144, one extending from each of the receptacle side walls 132 and 134. Springs 144 are located between the tabs 140 and the interior wall 136 in the illustrated embodiments. In other embodiments (not shown), the springs 144 may located at other positions, such as for example opposite the tabs 140 from the interior wall 136. As described in greater detail below, springs 144 are configured to help retain the connector plug 46 within the receptacle 120 when the connector plug is coupled to the connector socket 100.

Embodiments of the connector socket 100 include alignment structures 146 extending into the receptacle 120 from one or more of the receptacle walls 132, 134 and 136. The illustrated embodiments include a plurality (three are shown for purposes of example) of alignment structures 146 extending from the receptacle interior wall 136. Alignment structures 146 are generally rectangular in cross section in the illustrated embodiments, and taper to reduced cross sectional size at their ends adjacent to the first major wall 106 in the illustrated embodiments. As described in greater detail below, the alignment structures 146 cooperate with the connector plug 46 to guide the connector plug to its proper mating position in the receptacle 120 when the connector plug is coupled to the connector socket 100.

Connector socket 100 also includes one or more first electrical contacts 150 that are located on and/or extend from the contact wall 130. The illustrated embodiments of connector socket 100 include a plurality of first electrical contacts 150. The first electrical contacts 150 are spring-loaded pogo pin contacts that extend from the contact wall 130 into the receptacle 120 in embodiments. Other embodiments of the connector socket 100 include other types of electrical contacts.

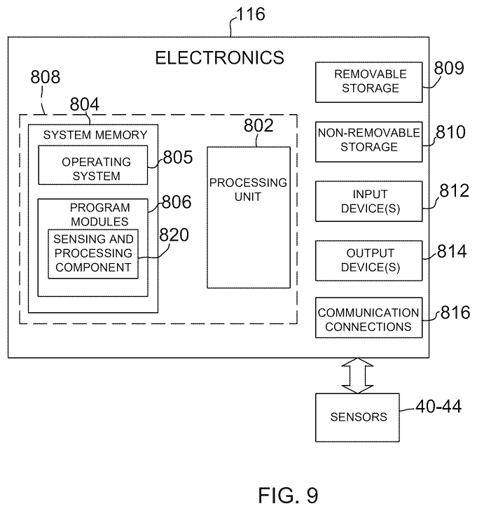

FIG. 9 is a block diagram illustrating exemplary physical components (e.g., hardware) of electronics 116 that may be incorporated into the electronic component 18 and communicatively coupled to the sensor harness 14 as described herein (e.g., to the sensors 40-44). In a basic configuration, the electronics 116 is configured as a computing device or controller with data interface capabilities and may include at least one processing unit 802 and a system memory 804. Depending on the configuration and type of computing device, the system memory 804 may comprise, but is not limited to, volatile storage (e.g., random access memory), non-volatile storage (e.g., read-only memory), flash memory, or any combination of such memories. The system memory 804 may include an operating system 805 and one or more, such as a sensing and processing component 820.

The operating system 805, for example, may be suitable for controlling the operation of the electronics 116. Furthermore, embodiments of the disclosure may be practiced in conjunction with a graphics library, other operating systems, or any other application program and is not limited to any particular application or system. This basic configuration is illustrated in FIG. 9 by those components within a dashed line 808. The electronics 116 may have additional features or functionality. For example, the electronics 116 may also include additional data storage devices (removable and/or non-removable). Such additional storage is illustrated in FIG. 9 by a removable storage device 809 and a non-removable storage device 810. Removable storage devices such as 809 may be used to transfer the information collected from the sensor harness 14 to other systems for use or analysis (e.g., applications or other programs operation on a computer system or a mobile device).

As stated above, a number of program modules and data files may be stored in the system memory 804. While executing on the processing unit 802, the program modules 806 (e.g., the sensing and processing component 820) may perform processes including, but not limited to, the aspects, as described herein, e.g., the data sensing aspects.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 9 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the electronics 116 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The electronics 116 may also have one or more input device(s) 812 such as visual image sensors, audio sensors, a sound or voice input device, a touch or swipe input device, etc. The output device(s) 814 such as a display, speakers, etc. may also be included. The aforementioned devices are examples and others may be used. The electronics 116 may include one or more communication connections 816 allowing communications with other computing devices, for example that process and analyze or otherwise use the data collected by the sensor harness 14 (e.g., applications or other programs operation on a computer system or a mobile device). Examples of suitable communication connections 816 include, but are not limited to, radio frequency (RF) transmitter, receiver, and/or transceiver circuitry; universal serial bus (USB), parallel, and/or serial ports. In other embodiments, the electronic component 18 may further comprise any one or more of the movement, position, physiological, and/or environmental data sensors described herein.

The term computer readable media as used herein may include computer storage media. Computer storage media may include volatile and nonvolatile, removable and non-removable media implemented in any method or technology for storage of information, such as computer readable instructions, data structures, or program modules. The system memory 804, the removable storage device 809, and the non-removable storage device 810 are all computer storage media examples (e.g., memory storage). Computer storage media may include RAM, ROM, electrically erasable programmable read-only memory (EEPROM), flash memory or other memory technology, optical storage, magnetic storage devices, or any other article of manufacture which can be used to store information, and which can be accessed by the electronics 116. Any such computer storage media may be part of the electronics 116. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Referring back to FIGS. 6-8, connector plug 46 includes a body 200 having a mating portion 202, an overmold portion 203, and one or more second electrical contacts 204 on the mating portion. A plurality of second electrical contacts 204 are shown in the illustrated embodiments. Cable 48 extends from the body 200, and each of the wires 50 of the cable (not visible in FIG. 7 or 8) is electrically coupled to at least one of the second electrical contacts 204, via a support member (not shown). In some embodiments, the connector plug comprises a support member, wherein the second electrical contacts are mounted to the support member. The mating portion 202 of the body 200 is configured to mate with the receptacle 120 of the connector socket 100 (e.g., is located, sized and shaped to fit within the receptacle), and is defined by a bottom wall 210, top wall 212 side walls 214 and 216, and end wall 218. The overmold portion 203 extends from a side of the mating portion 202 opposite the end wall 218, and is defined by a bottom wall 220, top wall 222, side walls 224 and 226, and an end wall 228. In the illustrated embodiments, the bottom wall 220, top wall 222 and side walls 224 and 226 of the overmold portion 203 extend in a generally co-planar relationships from the corresponding bottom wall 210, top wall 212 and side walls 214 and 216 of the mating portion 202. In other embodiments (not shown), the overmold portion 203 has different configurations. Yet other embodiments of the connector plug 46 (not shown) do not include the overmold portion 203. Connector plug 46 may be a unitary molded member. For example, the mating portion 202 can be formed from first and second polymer members (not separately shown) that are joined to one another to seal the second electrical contacts 204 within the body 200, and the overmold portion 203 can be formed over the body to cover the cable 48 and portions of the body to enhance the water proof characteristics of the body. In some embodiments, the connector plug 46 comprises a first polymer member, a second polymer member joined to the first polymer member with the support member sealed between the first and second polymer members; and an overmold portion sealed to and extending over at least portions of the first and second polymer members and wherein a portion of the cable extends from the overmold portion.

The second electrical contacts 204 of the connector plug 46 are positioned on the mating portion 202 and are configured to cooperate with the first electrical contacts 150 of the connector socket 100 to couple electrical signals from the sensor harness 14 to the electronics 106 of the electronic component 18 when the mating portion is received in the receptacle 120 of the connector socket. In embodiments, the second electrical contacts 204 of the connector plug 46 are in physical mechanical contact with the first electrical contacts 150 of the connector socket 100 when the connector plug 46 is coupled to the connector socket. In the illustrated embodiments, the second electrical contacts 204 are positioned on the bottom wall 210 of the mating portion 202. The second electrical contacts 204 may be electrically conductive members, such as for example metal members, optionally copper or gold-plated conductive members, that are fixedly mounted with respect to the bottom wall 210. For example, the material forming the mating portion 202 may physically contact at least the peripheral sides of the second electrical contacts 204 to provide a water resistant or waterproof seal between the mating portion 202 of the body and the electrical contacts 204. In the embodiments shown in FIG. 7, the contact surfaces of the second electrical contacts 204 are recessed below the bottom wall 210 of the mating portion 202. In other embodiments (not shown) the contact surfaces of the second electrical contacts 204 are generally flush or parallel with the bottom wall 210, or may extend beyond the bottom wall. Other embodiments of the connector plug 46 include other types and/or configurations of second electrical contacts 204.

The illustrated embodiments of the mating portion 202 of the connector plug 46 includes one or more tab recesses 230 (two are shown for purposes of example) on one or more of the side walls 214 and 216. Each of the tab recesses 230 is configured (e.g., located, sized and/or shaped) to receive and mate with one of the tabs 140 of the connector socket 100 when the connector plug 46 is coupled to the connector socket. Embodiments of the connector plug 46 configured for use with embodiments of the connector socket 100 that do not include tabs 140 may not have the tab recesses 230. In yet other embodiments (not shown), one or more tabs such as 140 can be located on the connector plug 46 (e.g., rather than on the connector socket 100), and a complementary mating tab recess such as 230 can be located on the connector socket (e.g., rather than on the connector plug).

The illustrated embodiments of the mating portion 202 of the connector plug 46 include one or more alignment recesses 232 (three are shown for purposes of example) on the end wall 218. Each of the alignment recesses 232 is configured (e.g., located, sized and/or shaped) to receive and mate with one of the alignment structures 146 of the connector socket 100 when the connector plug 46 is coupled to the connector socket. Embodiments of the connector plug 46 configured for use with embodiments of the connector socket 100 that do not include alignment structures 146 may not have the alignment recesses 232. In yet other embodiments (not shown), one or more alignment structures such as 146 can be located on the connector plug 46 (e.g., rather than on the connector socket 100), and a complementary mating alignment recess such as 232 can be located on the connector socket (e.g., rather than on the connector plug).

The illustrated embodiments of the mating portion 202 of the connector plug 46 includes one or more spring recesses 234 (two are shown for purposes of example) on one or more of the side walls 214 and 216. Each of the spring recesses 234 is configured (e.g., located, sized and/or shaped) to receive and mate with one of the springs 144 of the connector socket 100 when the connector plug 46 is coupled to the connector socket. Embodiments of the connector plug 46 may not have spring recesses 234, even if the connector socket 100 includes springs 144. Embodiments of the connector plug 46 configured for use with embodiments of the connector socket 100 that do not include springs 144 may not have the spring recesses 234. In yet other embodiments (not shown), one or more springs such as 144 can be located on the connector plug 46 (e.g., rather than on the connector socket 100), and a complementary mating spring recess such as 234 can be located on the connector socket (e.g., rather than on the connector plug).

Alternative or additionally, the connector socket 100 and/or the connector plug 46 may include other structures to enhance the strength of retention and to maintain the retention of the connector plug in the connector socket when these connector components are coupled to one another. For example, a pressure sensitive adhesive may be applied to one or more walls or other surfaces of the connector socket 100 and/or the connector plug 46 that contacts a wall or other surface of the other component. As another example, hook and loop fasteners may be incorporated into the connector socket 100 and the connector plug 46. In another embodiment, the electronic component 18 comprises a sliding lock (not shown) that can move from a first unengaged position (e.g., that is parallel or coplanar with the first major wall 106) to a second locking position that at least partially covers connector plug 46 when connector plug 46 and electronic component 18 are in a coupled position to help retain the connector plug 46 in the coupled position. In other embodiments, the connector plug 46 can be retained on the electronic component 18 using elastic bands, clips or other known retaining mechanisms.

The connector plug 46 and socket connector 100 are configured so that when they are coupled to one another, the mating portion 202 of the connector plug 46 is received within the receptacle 120 of the socket connector 100, and the second electrical contacts 204 of the connector plug are electrically coupled to, and in embodiments physically contact, associated first electrical contacts 150 of the connector socket 100. The bottom wall 210 of the connector plug 46 is located adjacent to, and in embodiments is generally parallel to, the bottom wall 130 of the connector socket 100 when the connector plug is coupled to the connector socket 100.

The connector plug 46 and connector socket 100 are connected or attached in the coupled relationship through movement of the connector plug with respect to the connector socket about a coupling path. Similarly, the connector plug 46 and connector socket 100 may be removed, disconnected or detached from the coupled relationship through movement of the connector plug with respect to the connector socket about the coupling path. The coupling path of the illustrated embodiments is a direction that extends generally through both the bottom wall 210 and the top wall 212 of the mating portion 202 of the connector plug 46. When the connector plug 46 and connector socket 100 are being connected, the connector plug leads with the bottom surface 210 during the motion along the coupling path, and effectively enters the receptacle 120 through an opening in the first major wall 106 of the housing 104. Similarly, the connector plug 46 trails with the bottom surface 210 during the motion along the coupling path when the connector plug 46 and the connector socket 100 are being disconnected. In some embodiments, the bottom wall 210 mates with bottom wall 130 when connected. In embodiments, where bottom wall 130 comprises a raised portion 131, the bottom wall 210 is shaped to fit the bottom wall 130 and raised portion 131 acts to retain the connector plug 46 from being removed in any direction that is not along the coupling path. For example, portions of mating portion 202 and/or overmold portion 203 may be recessed with respect to the bottom wall 210 to define a shoulder on the side of the mating portion opposite the end wall 218, and the shoulder is configured to engage the raised portion 131.

Motion about the coupling path, and the direction of the coupling path, may be guided by characteristics, such as the orientations, of one or more of the walls 132, 134 and 136, the alignment structures 146 and/or the tabs 140 and/or raised portion 131 of the connector socket 100, and/or the walls 212, 214 and 216, the alignment recesses 232 and/or the tab recesses 230 of the connector plug 46. In the illustrated embodiments, the walls 132, 134 and 136, alignment structures 146 and tabs 140 of the connector socket 100, and the walls 212, 214 and 216, alignment recesses 232 and tab recesses 230 of the connector plug 46 are generally perpendicular to the major planar surface of the first major wall 106 of the housing 104, and generally parallel with the side wall 108 of the housing. The illustrated embodiments of the connector plug 46 are therefore moved about a coupling path that is generally perpendicular to the first major wall 106 of the housing when the connector plug is being connected to and being disconnected from the connector socket 100. The alignment structures 146 cooperate with the alignment recesses 232 to guide and help ensure accurate positioning of the connector plug 46 in the connector socket 100 during the connection of connector plug to the connector socket. In addition to the corresponding and cooperating shapes and sizes of the mating portion 202 of the connector plug 46 and the receptacle 120 of the connector socket 100, the tabs 140 of the connector socket cooperate with the tab recesses 230 of the connector plug to help retain the connector plug in its coupled position with the connector socket. Similarly, the springs 144 of the connector socket 100 engage the side walls 214 and 216 of the connector plug 46, and cooperate with any spring recesses such as 234 of the connector plug, to help retain the connector plug in its coupled position with the connector socket.

As perhaps best shown in FIG. 8, when the connector plug 46 is coupled in the connector socket 100 of the electronic component 18, portions of the body 202 including at least portions of the overmold portion 203, extend from the receptacle 120 beyond the side wall 108 of the housing 104. In embodiments, portions of the mating portion 202 of the body 200 may also extend beyond the side wall 108 of the housing. The top wall 212 of the connector plug 46 is generally parallel with, and in embodiments generally flush or coplanar with, the first major wall 106 of the housing 104 of the electronic component 18. The illustrated configuration of the connector plug 46 and connector socket 100 resist movement and disconnection along any path with respect to one another that extends through the side wall 108 (e.g., in any direction ±90° with respect to a direction perpendicular to the side wall 108. In effect, the configuration of the connector plug 46 and connector socket 100 causes the connector plug to resist disconnection from the connector socket in response to forces in any direction other than the direction of the coupling path.

The cable 48 of the sensor harness 14 extends from the body 200 of the connector plug 46. In the illustrated embodiments the cable 48 extends from the overmold portion 203 of the body 200. In other embodiments (not shown), such as for example those without the overmold portion 203, the cable 48 extends from other portions of the body, such as the mating portion 202. In the illustrated embodiments, the cable 48 and wires 50 therein extends from the body 200 of the connector plug 46 in a direction that is generally perpendicular to the direction of the coupling path along which the connector plug is moved during the connection and disconnection of the connector plug with respect to the connector socket 100. By this configuration, disconnection of the connector plug 46 from the connector socket 100 is resistant to forces exerted on the connector plug by the cable 48 when the smart garment 10 is being worn by a user. In other embodiments, advantageous disconnection-resistant characteristics of the connector system can be provided when the cable 48 extends from the body 200 of the connector plug 46 at other angles, such as for example at an angle between 45° and 135° with respect to the coupling path.

Smart garment 10 can provide for the accurate and reliable collection of data from users wearing the garment. The data can be used for a variety of purposes, such as for ergonomic analysis (e.g., back and shoulder ergonomic analysis in shirt or jacket embodiments of the smart garment 10). The smart garment 10 may also be comfortable to wear, and have minimal or no substantial impact on the user's ability to perform routine or otherwise expected movement.

Prototypes have demonstrated capability of suitable performance following at least fifty 40° C. wash/warm tumble dry machine washes and one thousand and five-hundred mating cycles of the connector plug 46 and connector socket 100.

Data quality for ergonomic sensing may depend at least in part on knowledge of the sensor positions. Smart garments 10 provide these and other characteristics. The sensors remain fixed in position and/or orientation, and as close to the body as reasonably possible, to provide accurate motion and other data. The smart garment provides wear comfort, durability and hygienics, all with relatively low set-up time.

Embodiments of the smart garment 10 include a midlayer shirt, for example a long sleeve shirt or jacket made of performance fabric with an integrated electronic sensor harness that facilitates multi-point upper body motion capture. Embodiments of the sensor harness are comprised of an electronic webbing (e.g., wires knitted into an elastic textile trim), one or more, such as for example five, satellite sensors devices (e.g., each containing one or more of an accelerometer, a gyroscope and/or a magnetometer), and a connector. The sensor harness may be T-shaped and electrically connects and positions the satellite sensors (e.g., two on each arm, such as at the wrist and bicep, and one at the neck). The sensors may be permanently attached (e.g., soldered) to the electronic webbing and encapsulated for waterproofing against washing. The encapsulation of the sensors and the attachment points can help to provide a water resistant or waterproof harness to the smart garment 10. The connector plug 46 may terminate at the bottom of the “T” and facilitates connection to an electronic component such as a controller. The electronic component may provide control, power supply and other functionality, and may also include a sensor (e.g., for sensing motion). The sensor harness may be attached to the garment, for example to the inside of the garment, by stitching or other approaches/structures, and may run along the back of the arms and down the spine. The sensors may be covered by textile badges to minimize or prevent interaction with the user's skin. In embodiments where the sensor harness is located on the inside of the garment, these electronic components may be essentially invisible. The attachment of the sensor harness to the garment may provide for ease of removal at end of life. The connector of the harness may reside in a pocket or be attached to the garment by other attachment structures, and may be located on a tighter-fitting elastic waistband or other portion of the garment, for example the back hip. To attach the controller, a user may pull the connector from the pocket, attach it to the controller, and store the controller (and in embodiments the connector) in the pocket. The connector and pocket may be configured for convenient user access, and the pocket may relatively tightly hold the controller so that accurate data, such as for example motion, physiologic or other data from the user's pelvis can be captured, and to reduce the likelihood that the controller is disconnected from the sensor harness or garment.

The electrical connector system facilitates both electrical connection and mechanical connection. By the electrical connection, power such as low voltage current can flow across the connector in a number of distinct channels. By the mechanical connection the removable component is physically bound to the rest of the system at the discretion of the user. The connection may be relatively easy for the user to bind and unbind, but present a relatively low risk of unintentional separation. The connector system exhibits a number of important characteristics. For example, the garment-side of the connector is able to withstand the care regime of the garment, such as for example mechanical stresses, heat, detergent, fouling, or corrosion from the washing process. The connector is large enough to be handled by a wide range of users, but not so large that it is uncomfortable to wear. The connector system is sufficiently robust to resist stresses of wear. For example, when the user is walking or otherwise moving through a range or anticipated motions during normal use, the stresses will not cause an unacceptable loss of connection. The connector system is relatively fool-proof and intuitive to use. It may be efficient to manufacture.

The contacts of the connector plug may be relatively large and flush with the body. Resistance to fouling and corrosion during washing is thereby provided (e.g., as opposed to connectors with recesses or openings that may become clogged by debris during washing). The connector plug and connector socket may be coupled in only one orientation or way. Risks associated with accidental incorrect polarity connections may be alleviated. Embodiments with the 90-degree elbow shape may result in reduced dimensions of the connector plug and reduce the overall dimensions of the connector system when joined to the connector socket. Risk of accidental disconnection may be reduced, for example if the shirt-side cable is pulled in-line with the electronic component. The tabs or lugs help maintain the retention, and also help prevent unintentional disconnection. Use of spring-loaded pin connectors helps ensure robust electrical contacts, for example though vibrations and impacts to which the electronic component might be subjected during use. The springs, which may be leaf springs, may be configured to counteract forces provided by the spring-loaded pin connectors, and can be adjusted or replaced as needed. Embodiments of the connector plug that contain no moving or sensitive parts provide wash durability. The profile of the connector plug is flush and minimalistic. The overmold portion may provide stress relief or tapering between the connector plug and other components of the sensor harness. The connector plug may be potted, for example with hot-melt compound, to help ensure waterproofness and integrity. In embodiments, only relatively robust components are located in the connector plug to enhance durability though wash cycles.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A smart garment, comprising:
a garment configured to be worn by a user, comprising:
a trunk portion and first and second limb portions extending from the trunk portion, wherein the first limb portion is configured to cover at least a portion of a first limb of the user, and the second limb portion is configured to cover at least a portion of a second limb of the user; and
an electronic component attachment structure configured to receive an electronic component in a releasable manner to a location on the trunk portion, wherein the electronic component attachment structure comprises a pocket and the pocket opens to an interior or an exterior of the trunk portion; and
wherein the trunk portion comprises:
a first portion configured to extend around a trunk of the user when worn, and wherein the first portion has a first elasticity in a circumferential direction; and
a second portion attached to the first portion and configured to extend around the trunk of the user when worn, wherein the second portion has a second elasticity in a circumferential direction that is greater than the first elasticity, and wherein the electronic component attachment structure is in or on the second portion of the trunk portion; and
a sensor harness attached to the garment, including:
a plurality of wires;
an electrical connector component coupled to the plurality of wires, wherein the electrical connector component is configured to be mechanically and electrically coupled in a releasable manner to an electronic component attached to the garment by the electronic component attachment structure; and
a plurality of sensors, including at least a first sensor, a second sensor, and a third sensor coupled to the plurality or wires, wherein the first sensor is located on the first limb portion, the second sensor is located on the second limb portion, and the third sensor is located on the trunk portion.

2. The smart garment of claim 1, wherein the electronic component attachment structure comprises one or more of a pocket, a tunnel, a snap, a hook and loop fastener, a mount, stitching, gluing, lamination, welding, or a combination thereof.

3. The smart garment of claim 1, wherein the electronic component attachment structure comprises a pocket and the pocket opens to an interior or an exterior of the trunk portion.

4. The smart garment of claim 1, wherein:
the electronic component attachment structure comprises a tunnel, the tunnel comprising two openings; the first opening to an exterior of the trunk portion; and
a connector opening that opens to the interior of the trunk portion,
wherein a portion of the plurality of wires and the electrical connector component extend through the connector opening into the tunnel.

5. The smart garment of claim 4, wherein the connector opening opens into the tunnel.

6. The smart garment of claim 1 comprising a sensor cover that is configured to cover over one or more of the plurality of sensors on an interior of the garment.

7. The smart garment of claim 6, wherein the sensor cover is configured to attach the associated one or more of the plurality of sensors to the garment.

8. The smart garment of claim 6, wherein the sensor cover defines a tunnel in which the associated one or more of the plurality of sensors is located.

9. The smart garment of claim 1, comprising a harness cover that is configured to cover over at least a portion of the plurality of wires.

10. The smart garment of claim 9, wherein the harness cover defines a tunnel in which the associated portions of the plurality of wires are located.

11. (canceled)

12. The smart garment of claim 1, wherein the trunk portion comprises:
a first portion, configured to extend around a trunk of the user when worn, and wherein the first portion has a first diameter when in an unstretched state; and
a second portion attached to the first portion and configured to extend around the trunk of the user when worn, wherein the second portion has a second diameter that is less than the first diameter when in an unstretched state, and wherein the electronic component attachment structure is on the second portion of the trunk portion.

13.-16. (canceled)

17. The smart garment of claim 1, wherein the sensor harness comprises:
a first torso portion extending transversely across the trunk portion, across and between shoulders of the user when the garment is worn, and wherein the third sensor is on the torso portion of the sensor harness;
a second torso portion extending longitudinally down the trunk portion from the first torso portion, and wherein the electrical connector component is located on an end of the second torso portion opposite the first torso portion;
a first sleeve portion extending from the first torso portion onto the first sleeve, and wherein the first sensor is on the first sleeve portion; and
a second sleeve portion extending from the first torso portion onto the second sleeve, and wherein the second sensor is on the second sleeve portion.

18. The smart garment of claim 1, wherein the sensor harness includes:
a torso portion extending transversely across the trunk portion of the garment, and wherein the third sensor is on the torso portion of the sensor harness;
a first limb portion extending from the torso portion onto the first limb portion of the garment, and wherein the first sensor is on the first limb portion; and
a second limb portion extending from the torso portion onto the second limb portion of the garment, and wherein the second sensor is on the second limb portion.

19.-20. (canceled)

21. The smart garment of claim 1, further comprising an electronic component attached to the garment by the electronic component attachment structure and coupled to the sensor harness via the electrical connector component.

22. (canceled)

23. The smart garment of claim 1, wherein the garment is configured to be machine washable when the sensor harness is attached to the garment, and no electronic component is received by the connector plug.

24. The smart garment of claim 1, wherein the garment is configured to facilitate removal of the sensor harness from the garment.

25. The smart garment of claim 1, wherein at least portions of the sensor harness are not visible from an exterior of the garment.

26. A smart garment comprising:
a garment configured to be worn by a user, comprising:
a trunk portion and first and second limb portions extending from the trunk portion, wherein the first limb portion is configured to cover at least a portion of a first limb of the user, and the second limb portion is configured to cover at least a portion of a second limb of the user; and
an electronic component attachment structure configured to receive an electronic component in a releasable manner to a location on the trunk portion, wherein the electronic component attachment structure comprises a pocket and the pocket opens to an interior or an exterior of the trunk portion; and
a sensor harness attached to the garment, including:
a flexible base;
a plurality of wires;
an electrical connector component coupled to the plurality of wires, wherein the electrical connector component is configured to be mechanically and electrically coupled in a releasable manner to an electronic component attached to the garment by the electronic component attachment structure; and
a plurality of sensors, including at least a first sensor, a second sensor, and a third sensor coupled to the plurality or wires, wherein the first sensor is located on the first limb portion, the second sensor is located on the second limb portion, and the third sensor is located on the trunk portion
wherein the plurality of wires is attached to the flexible base,
wherein the trunk portion comprises a first portion configured to cover an upper portion of the torso, and a second portion configured to cover a lower portion of the torso; and
wherein the electrical attachment structure is located on the second portion.
